(19) United States
(12) Patent Application Publication
(10) Pub. No.: US 2010/0241118 A1
(43) Pub. Date: Sep. 23, 2010

(54) HIGH FREQUENCY TREATMENT INSTRUMENT

(75) Inventor: Hidefumi AKAHANE, Saitama (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: FUJIFILM Corporation, Tokyo (JP)

(21) Appl. No.: 12/716,627

(22) Filed: Mar. 3, 2010

(30) Foreign Application Priority Data
Mar. 18, 2009 (JP) 2009-065486

(51) Int. Cl. A61B 18/14

(52) U.S. Cl. 606/41

(57) ABSTRACT

A high frequency treatment instrument is disclosed which comprises a flexible sheath having an axial passage hole, an operating means connected to a proximal end portion of said flexible sheath, a treatment instrument main body consisting of a flexible cord and an electrode provided at a distal end of the flexible cord to be inserted into the axial passage hole of the flexible sheath, the electrode being displaced between a retracted position in the flexible sheath and a protruded position out of a distal end by push or pull operation of the flexible cord by the operating means, the flexible cord consisting of a conductive body covered by an electrical insulating body and the electrode being electrically connected to the conductive body, and a lateral opening formed at a lateral surface of the flexible sheath, whereby a part of the electrode being exposed in the lateral opening at the retracted position, while the electrical insulating member of the flexible cord being faced with the lateral opening at the protruded position.

### BACKGROUND OF THE INVENTION

1. Field of the Invention

The present invention relates to a high frequency treatment instrument inserted into, for example, a body cavity through a guide means such as a biopsy channel of an endoscope and suitable for performing treatments such as dissection and ablation of a mucous membrane and the like.

2. Description of the Related Art

If a lesion such as a tumor is discovered in a mucous portion in a body-cavity inner wall of an esophagus in an endoscopic inspection for a gaster, a duodenum, a colon or the like, a treatment to ablate the lesion mucous membrane is performed. One of such treatments is a treatment called Endoscopic Submucosal Dissection (ESD). In order to perform the ESD treatment, in the first place, a lesion mucous membrane in the mucous membrane to carry out a dissection is marked, and bulging the portion including the lesion mucous membrane by local injection. Then, a mucous membrane of a portion is dissected along the line with an outer periphery side of the marked region by means of a high frequency treatment instrument. After then, the submucosal layer is removed the mucous membrane from a muscle layer by a dissection.

The high frequency treatment instrument used in the ESD treatment is constituted by connecting a flexible sheath to an operating means and inserting a flexible cord provided with a rod-shaped electrode at a distal end into this flexible sheath. A push/pull operating member of the operating means is connected to the flexible cord at its proximal end. This push/pull operating member is designed so as to manual operation to move the flexible cord in the flexible sheath. As a result, the electrode displaces between a retracted position in the flexible sheath and protruded position a distal end of the flexible sheath.

The dissection should be removed the mucous membrane (or the mucous membrane and a submucosal layer) without invading a muscle layer located under the submucosal layer. For that purpose, the electrode should be limited the length protrusion from the flexible sheath at the time of the dissection. On the other hand, because of the mucous ablation cutting off fibers in the submucosal layer between the mucous membrane and the muscle layer, the flexible sheath should be swing right and left efficiently. For this purpose, the length of the electrode to be protruding from the distal end of the flexible sheath is preferably longer than the dissection treatment. Therefore, in the ESD treatment, it is preferable to change the protruding length of the electrode between the dissection and the ablation. For example, Japanese Unexamined Patent Application Publication No. 2008-386 (hereinafter referred to as Patent Document 1) discloses a high frequency treatment instrument whose protruding length from the flexible sheath is longer in ablation than in dissection.

The high frequency treatment instrument in Patent Document 1 has a high frequency knife inserted in a flexible sheath. The high frequency knife has two electrode members, i.e. a first electrode member made of a rod-shaped member with a spherical tip end and a second electrode member made of a cylindrical shape slidably fitted into the first electrode member. The first electrode member and the second electrode member move integrally up to projecting from the flexible sheath by a predetermined length. When the high frequency knife protruded from the distal end of the flexible sheath by the predetermined length, the high frequency treatment instrument is able to perform the dissection. The length of the electrodes from the flexible sheath is limited to penetration of a mucous membrane but not reaching to a muscle layer through a submucosal layer. The second electrode member is restricted the protrusion at the position being brought into contact with a stopper ring provided on the flexible sheath for performing the dissection. On the other hand, by pressing the first electrode member, it can further project from a stopped position of the second electrode member by the predetermined length. Therefore, the high frequency knife becomes longer by a portion that the first electrode member projects from the second electrode member, which realizes to perform efficient mucous ablation.

With the construction as Patent Document 1, the two types of operations or treatments, that is, mucous dissection and ablation using a single high frequency treatment instrument can be performed smoothly and efficiently. However, the high frequency treatment instrument in Patent Document 1 is still encountered a problem to be solved. That is, this high frequency treatment instrument has to install two types of the electrode members. Moreover, the high frequency treatment instrument has a complicated mechanism to move both electrode members in conjunction with each other and to move the first electrode member singularly. In detail, the first electrode member needs to be slidable with respect to the second electrode member, and the stopper ring needs for regulating a projecting end of the second electrode member at the distal end of the flexible sheath. Moreover, a member for push/pull operating the first electrode member and a member for push/pull operating the second electrode member need separately, and the configuration of the push/pull operating members are also complicated.

### SUMMARY OF THE INVENTION

In view of the above points, it is an object of the present invention to enable two types of treatments or operations of mucous dissection and mucous ablation with a simple configuration.

Another object of the present invention is to enable displacement of a high frequency knife between a state for performing mucous dissection and a state to perform mucous ablation by an operation of single operating means.

According to the present invention, in order to achieve the above-mentioned objects, there is provided a flexible sheath having an axial passage hole, an operating means connected to a proximal end portion of said flexible sheath, a treatment instrument main body consisting of a flexible cord and an electrode provided at a distal end of said flexible cord to be inserted into said axial passage hole of said flexible sheath, said electrode being displaced between a retracted position in said flexible sheath and a protruded position out of a distal end by push or pull operation of said flexible cord by said operating means, said flexible cord consisting of a conductive body covered by an electrical insulating body and said electrode being electrically connected to said conductive body, and a lateral opening formed at a lateral surface of said flexible sheath, whereby a part of said electrode being exposed in said lateral opening at said retracted position, while said electrical insulating member of said flexible cord being faced with said lateral opening at said protruded position.

A conventional high frequency treatment instrument constitutes generally that an electrode of a rod shape is provided inside a flexible sheath and a flexible cord is connected to a proximal end portion of the electrode so that the electrode goes in and out of the flexible sheath by pull and push operation of the flexible cord. The high frequency treatment instrument is adapted to insert into a biopsy channel of an endoscope, and the flexible sheath has an outer diameter adapted to insert into the biopsy channel. Further, the flexible cord is capable of being bent, and such a cord with an inside being a conductive member communicating with the electrode and an outer face covered with an insulating coating of fluorine resin or the like. An operation means is connected for manually push and pull the electrode through the flexible cord at the proximal end portion of the flexible sheath, and the electrode moves between an operating position protruded from the flexible sheath and a receded position into the flexible sheath by this operating means in general.

If the electrode is located at the operating position, the entirely protruded portion from the flexible sheath including the tip end portion of the electrode has a function as an electrode member for performing a treatment, and mucous can be dissected. On the other hand, even at the receded position, the electrode has a function as an electrode member. That is, at a portion of the side surface of the electrode is exposed on the lateral opening of the flexible sheath, ablation of a mucous membrane dissected by exposed outer surface of the electrode t is performed by the rod-shaped electrode. Therefore, a high frequency current from a power source device should cut off when the electrode is connected while the treatment is not performed, but the high frequency current is exerted through the electrode when a treatment of mucous ablation is performed. Here, a width dimension of the lateral opening is smaller than an outer diameter dimension of the electrode and a length dimension is shorter than a length of the electrode.

More preferably, the width dimension of the lateral opening is slightly smaller than the outer diameter of the electrode, and the length of the lateral opening is slightly shorter than the entire length of the electrode in the axial direction. However, in order to dissect the mucous membrane, the high frequency current is necessary to flow through the electrode, but in order to limit a piercing depth in dissection, the maximum length of the electrode when protruded from the flexible sheath needs to be limited. On the other hand, in the treatment of mucous ablation or the like by exposing the electrode from the lateral opening, its dimension in the length direction is preferably long to some extent. In order to satisfy the above demands as mentioned above, not only a relation between the length of the electrode and the length of the lateral opening in the axial direction but also an opening position of the lateral opening should be adjusted.

Even a part of the electrode should not appear at the lateral opening when projecting from the flexible sheath as the operating position. To have sufficient length for the electrode, the lateral opening should keep away as far as possible from the distal end of the flexible sheath at the operating position. Therefore, the both of the electrode and the lateral opening can have sufficient length for performing the electrode ablation at the receded position. However, elongation of the electrode and the lateral opening will be encountered difficulty in the occasion of the treatment such as mucous ablation performed after dissection becomes poor. Therefore, the position and the length in the axial direction of the lateral opening need to be set properly, considering operability.

Anyway, at the operating position, the electrode needs to protrude from the flexible sheath by a predetermined length and to be positioned slightly in front of the distal end position of the lateral opening at the receded position, and a movable stroke needs to be limited. For this purpose, a positioning means of the distal end portion of the flexible sheath or the operating means is provide to restrict the treatment instrument main body. The positioning means can be a stopper means provided at a border portion between the flexible cord and the electrode, and a throttle means with which the both locking portions is brought into contact at positions front and rear of the lateral opening in the flexible sheath. Further, in the operating means can be provided for a moving stroke limiting means with which the moving stroke of the electrode can be limited between the retracted position and the projecting position.

A passage through which the flexible cord and the electrode provided at the distal end thereof are inserted is formed in the flexible sheath and is preferably configured such that a washing liquid can be injected from the distal end of the flexible sheath. For that purpose, provided in the flexible sheath is provided a liquid feeding passage, but this liquid feeding passage is preferably to provide separately from the passage for inserting the high frequency treatment instrument. Therefore, in the flexible sheath, two passages are formed, both extending from the flexible sheath to the portion on the proximal end side. For example, a position of a liquid supply port is between the flexible sheath and the operating means in a connection member to connect the liquid feeding passage to the liquid supply port.

### BRIEF DESCRIPTION OF THE DRAWINGS

Hereinafter describes embodiments of the present invention with reference to the attached drawings. The present invention is not limited to the following embodiments.

In the drawings:

FIG. 1 is an entire configuration diagram of a high frequency treatment instrument showing an embodiment of the present invention;

FIG. 2 is an enlarged sectional view of an essential part of FIG. 1;

FIG. 3 is an appearance view of a distal end portion of the high frequency treatment instrument;

FIG. 4 is an X-X sectional view of FIG. 3;

FIG. 5 is an explanatory diagram of a configuration showing the high frequency treatment instrument inserted into a biopsy channel of an endoscope;

FIG. 6 is an explanatory diagram of an action showing a section of the distal end portion of the high frequency treatment instrument at an operating position;

FIG. 7 is an explanatory diagram of an action a mucous membrane dissected using the high frequency treatment instrument;

FIG. 8 is an explanatory diagram of an action showing a section of the distal end portion of the high frequency treatment instrument at a receded position;

FIG. 9 is an explanatory diagram of an action showing to performing mucous ablation using the high frequency treatment instrument;

FIG. 10 is an explanatory diagram of an action showing in an enlarged manner a state in which the high frequency treatment instrument is inserted into a flexible sheath provided with positioning means and brought to the operating position;

FIG. 11 is an explanatory diagram of an action of a state in which the positioning means in FIG. 10 is at the receded position; and

FIG. 12 is an explanatory diagram similar to FIG. 8 to show another restricting means of the present invention.

### DESCRIPTION OF THE PREFERRED EMBODIMENTS

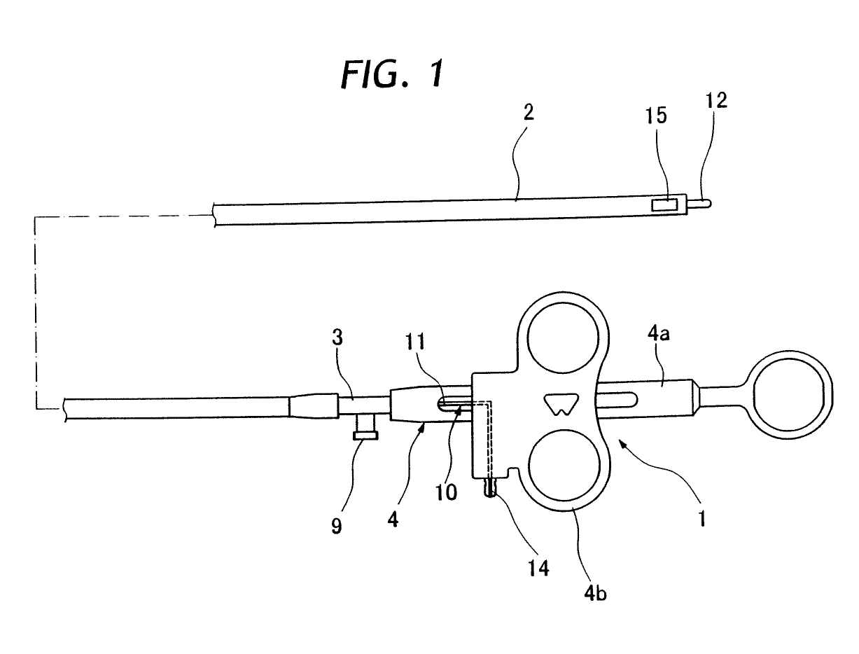

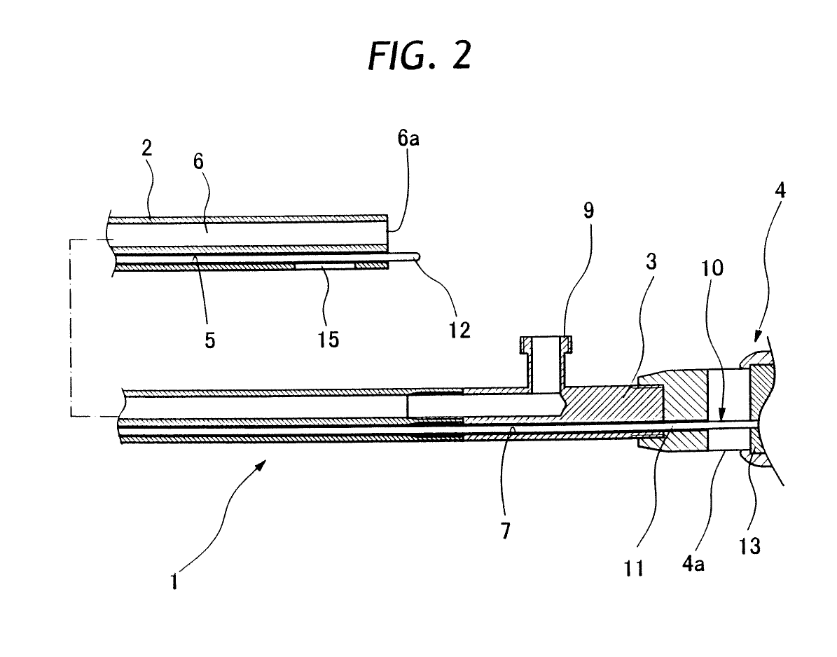

An embodiment of the present invention will be described based on the attached drawings. First, FIG. 1 shows an entire configuration of a high frequency treatment instrument and FIG. 2 shows an enlarged section of its essential part. In these figures, reference numeral 1 denotes a high frequency treatment instrument. The high frequency treatment instrument 1 has a elongated flexible sheath 2 fitted thereinto of a proximal end by a connecting member 3. An operating member 4 is connected to a side portion of a proximal end of the connecting member 3 by screwing. The operating means 4 is constituted by a main body shaft 4a connected to the connecting member 3 and a slider 4b slidably provided in the axial direction of the main body shaft 4a by fitting.

In the flexible sheath 2, an axial passage hole 5 and a fluid passage 6 are formed inside, and the axial passage hole 5 and the fluid passage 6 are provided penetrating through the flexible sheath 2. The proximal end portion of the flexible sheath 2 communicates to the connecting member 3, and the other end portion of the connecting member 3 is connected the main body shaft 4a of the operating means 4. The connecting member 3 is a member for connecting the flexible sheath 2 and the operating means 4, and in this connecting member 3, first and second passages 7 and 8 are formed. The first passage 7 extends, penetrating through the connecting member 3. The second passage 8 is bendable at 90 degrees inside the connecting member 3 and opened as a liquid supply port 9 in a side face.

The axial passage hole 6 formed in the flexible sheath 2 is opened at the distal end, and a treatment instrument main body 10 is inserted through the inside thereof. The treatment instrument main body 10 is constituted by a flexible cord 11 and an electrode 12 formed in a shape of a rod constituting a high frequency knife connected to the distal end of the flexible cord 11. The flexible cord 11 is constituted an outer periphery of a conductive wire covered by an electrical insulating member through coating with a fluorine resin or the like or otherwise the conductive wire is covered with an electrical insulating tube. The proximal end portion of the flexible cord 11 is lead out of the axial passage hole 5 and connected to a slide piece 13 connected to a slider 4b through the first passage 7 of the connecting member 3. The flexible cord 11 extends from the slide piece 13 to the slider 4b. Further, a contact portion 14 is lead out on a side face of the slider 4b. By moving the slider 4b in the operating means 4 along the main body shaft 4a, the slide piece 13 moves in the axial direction, and the electrode 12 goes in and out of the distal end of the flexible sheath 2. A connector from a high frequency power source, not shown, is detachably connected to the contact portion 14. As a result, a high frequency current is exerted through the electrode 12 disposed at the distal end of the flexible cord 11.

The fluid passage 6 provided in the flexible sheath 2 is opened as a liquid injection port 6a at the distal end of the flexible sheath 2. To the fluid passage 6, the second passage 8 of the connecting member 3 is connected, and this second passage 8 communicates with the liquid supply port 9 provided in the side face of the connecting member 3. Therefore, by connecting liquid supply means to the liquid supply port 9 and by pressure-feeding the liquid from the liquid supply means, a liquid such as a washing liquid is injected toward into the body cavity from the liquid injection port 6a through the fluid passage 6.

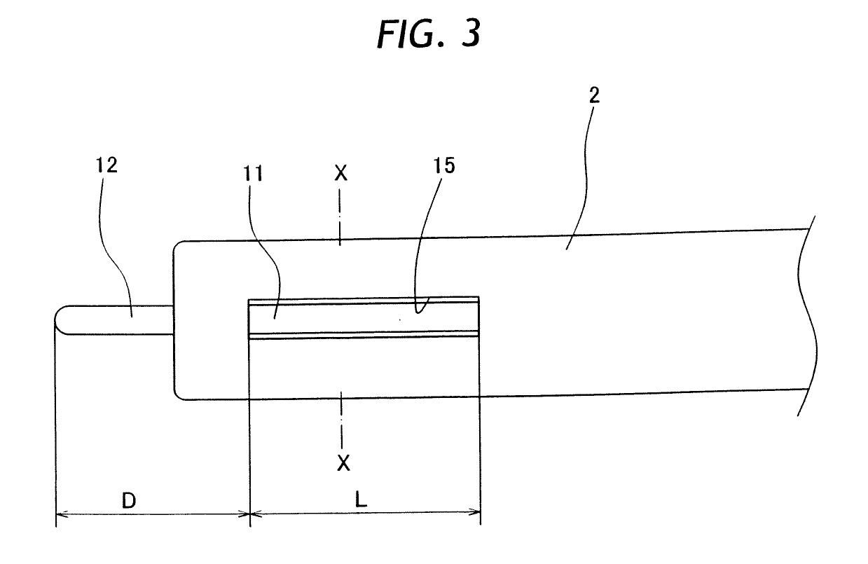

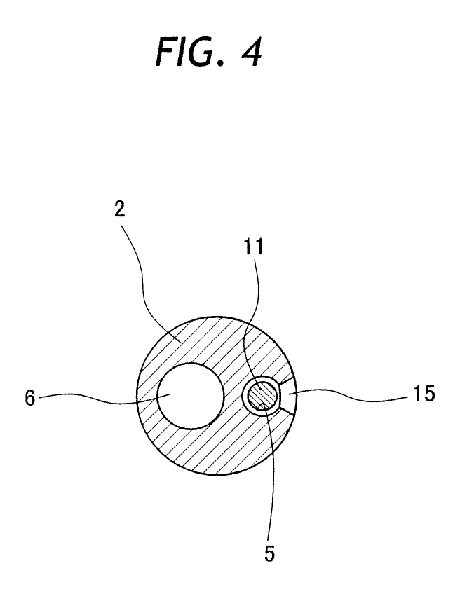

In the side face portion in the vicinity of the distal end of the flexible sheath 2, as shown in FIGS. 3 and 4, a lateral opening 15 for exposing the electrode is formed. This lateral opening 15 is an opening portion with a predetermined width and longitudinal length in the axial direction on the side face of the flexible sheath 2 and communicates with the axial passage hole 5. The opening width on the inserting path 5 side of the lateral opening 15 is smaller than a diameter dimension of the electrode 12 and preferably a dimension slightly smaller than the diameter dimension. If the width dimension of the lateral opening 15 is larger than the diameter dimension of the electrode 12, there is a fear that the electrode 12 might escape from the lateral opening 15. The most protruded position from the distal end position of the electrode 12 is the position as shown in FIG. 3, the length of the electrode 12 should be shorter than an interval D for placing the proximal end of the electrode 12 between the distal end of the electrode 12 and the end portion on the lateral opening 15. As a result, if the electrode 12 is located in the operating position, the flexible cord 11 of insulated is placed at the portion of the lateral opening 15, and when the high frequency current is made to flow through the electrode 12, the current will not leak from the portion of the lateral opening 15.

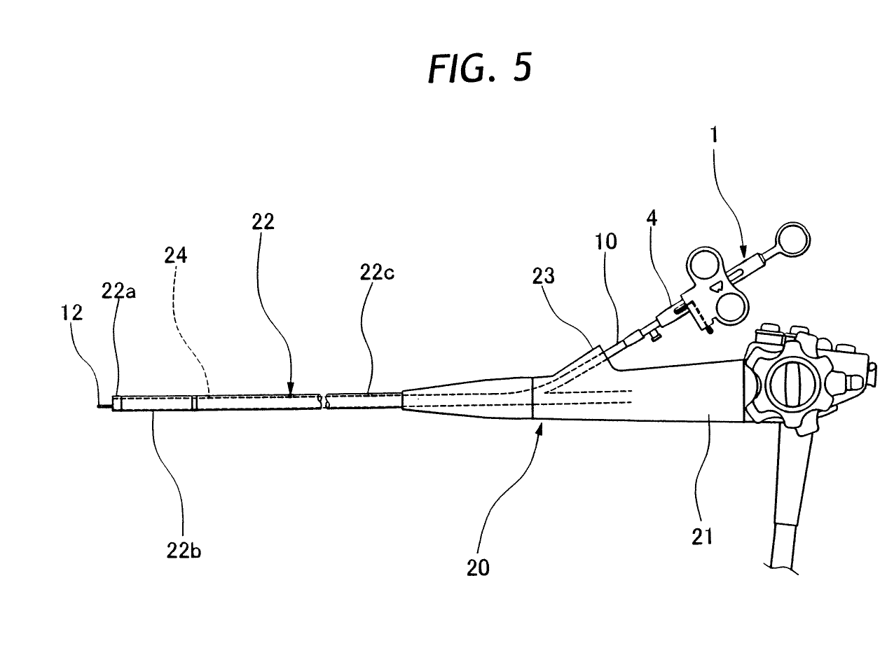

The high frequency treatment instrument 1 having the above configuration is, as shown in FIG. 5, inserted into a body cavity such as an esophagus, a gaster, a duodenum, a colon and the like through an endoscope 20 so as to examine whether or not a lesion in a mucous portion constituting a body cavity inner wall is present. If a lesion portion is discovered, the lesion mucous portion is specified as a treatment target portion, and a treatment to ablate only the treatment target portion, that is, an ESD treatment is performed. An endoscope 20 is provided by connecting an insertion portion 22 inserted into the body cavity with a main body operation portion 21. The insertion portion 22 has a flexible portion 22a for a predetermined length from the connection portion to the main body operation portion 21, a bent portion 22b at a distal end of the flexible portion 22a, and a distal-end hard portion 22c at a distal end of the bent portion 22b. At the distal-end hard portion 22c, endoscopic observing means including an illumination portion and an observation portion is attached as known. Also, a treatment instrument introduction portion 23 is provided on the main body operation portion 21, and a biopsy channel 24 is provided from the treatment instrument introduction portion 23 to a distal end face of the insertion portion 22.

The ESD treatment is to remove the lesion mucous membrane in the body cavity, and the high frequency treatment instrument 1 used for that purpose is inserted into the body cavity using the biopsy channel 24 provided in the endoscope 20 as guide means. When the ESD treatment is performed, damage on a healthy tissue should be avoided as much as possible and the lesion tissue should be fully removed. For that purpose, on a mucous membrane where the lesion portion to be ablated is present, that portion is marked to surround the lesion mucous region. After that, a saline solution is locally injected to the inside of the lesion mucous region. The local injection is made by using local injection means, not shown, in which a syringe needle is attached to the distal end of the flexible tube, and the local injection means is inserted through the biopsy channel 24.

By means of the local injection, the lesion mucous region is swollen by which the subsequent treatments such as mucous dissection and ablation to be performed after that can be conducted smoothly and safely. The making is performed by bringing a portion in a submucosal layer into a swollen state, and thus, there is no fear that the treatment to ablate the mucous membrane with the high frequency treatment instrument 1 damages the muscle layer or the like.

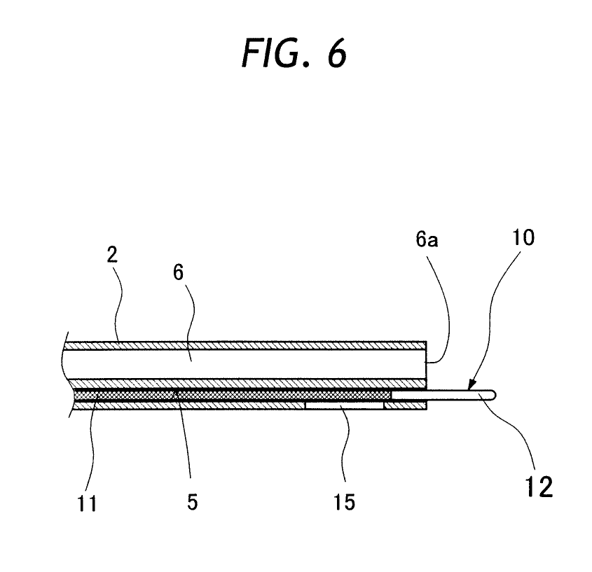

Subsequently, the lesion mucous membrane is dissected. For that purpose, the flexible sheath 2 of the high frequency treatment instrument 1 is inserted through the biopsy channel 24 of the endoscope 20, and the distal end portion of the flexible sheath 2 is led out of the biopsy channel 24 by a predetermined length. In order to perform dissection, by operating the operating means 4, the treatment instrument main body 10 inserted through the flexible sheath 2 is, as shown in FIG. 6, set at the protruded position where the electrode 12 provided at the distal end of the flexible cord 11 protrudes from the distal end of the flexible sheath 2. This is the operating position of the electrode 12. Here, in FIG. 6, the portion applied with electrical insulation processing on the outer face is shown by shading in the treatment instrument main body 10. Therefore, the rod portion on the distal end side from the shaded portion is the electrode 12.

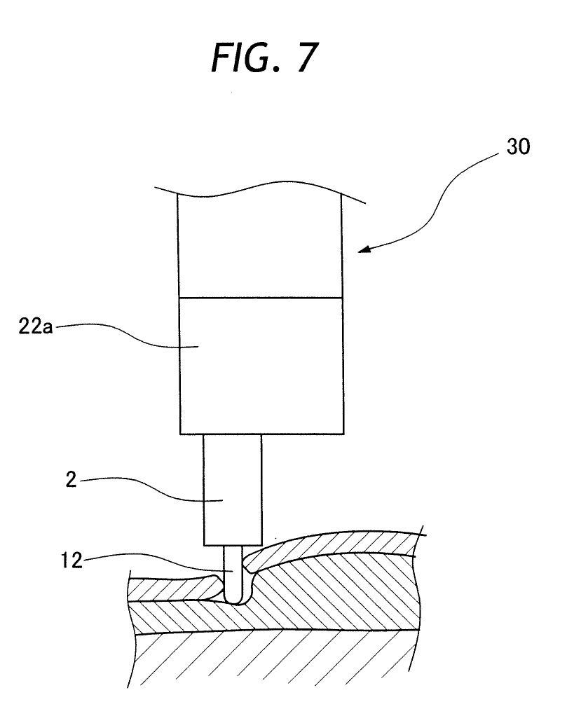

In this state, as shown in FIG. 7, by flowing the high frequency current from the external high frequency power source to the contact portion 14 provided on the operating means 4, the portion of the mucous layer is dissected. Here, the projecting length of the electrode 12 from the distal end of the flexible sheath 2 is a length such that when the electrode 12 pierces the mucous membrane, the distal end of the electrode 12 penetrates the mucous layer and reaches the submucosal layer but does not reach the muscle layer located beneath the submucosal layer. Actually, the dissecting portion is swollen by local injection, but the swollen portion is substantially a portion in the submocusal layer, and a thickness of the mucous membrane is hardly increased. Since the length of the electrode 12 is larger than the thickness of the mucous membrane, at least the mucous membrane can be dissected. Therefore, the projecting length of the electrode 12 is not made longer than necessary to allow for this torose state.

The dissection of the lesion mucous region is performed under observation of the electrode 12 by the observation portion of the endoscope by moving the insertion portion 22 of the endoscope 20 in a desired direction and by moving the distal end of the high frequency treatment instrument 1 as appropriate using the marked portion as a clue. By exerting the high frequency current through the electrode 12, the lesion mucous region in contact with the electrode 12 is cauterized and dissected. At this time, the treatment instrument main body 10 is partially faced with the lateral opening 15, but since this is the portion of the flexible cord 11 coated with the insulating member, there is no fear that the high frequency current leaks.

As mentioned above, the mucous membrane is dissected by moving the electrode 12 along the outside of the marked lesion mucous region. However, the mucous membrane cannot be ablated only by the dissection of the entire periphery of the mucous membrane. That is, since the mucous layer is linked with the muscle layer through the fibrous submucosal layer, the mucous ablation is performed by ablating the fibers. This mucous ablation can be also performed with the high frequency treatment instrument 1. Moreover, the ablation is performed with the high frequency treatment instrument 1 inserted in the biopsy channel 24 in the endoscope 20.

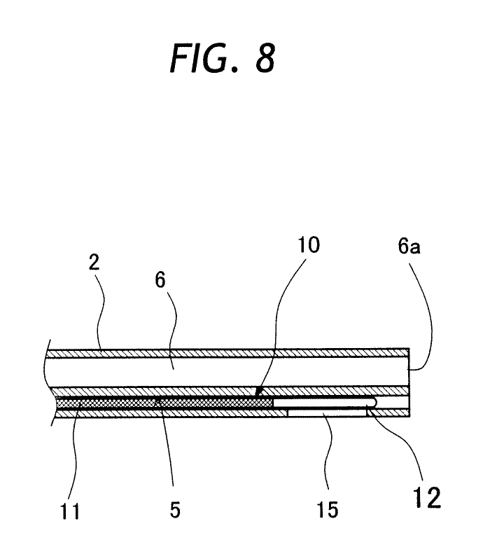

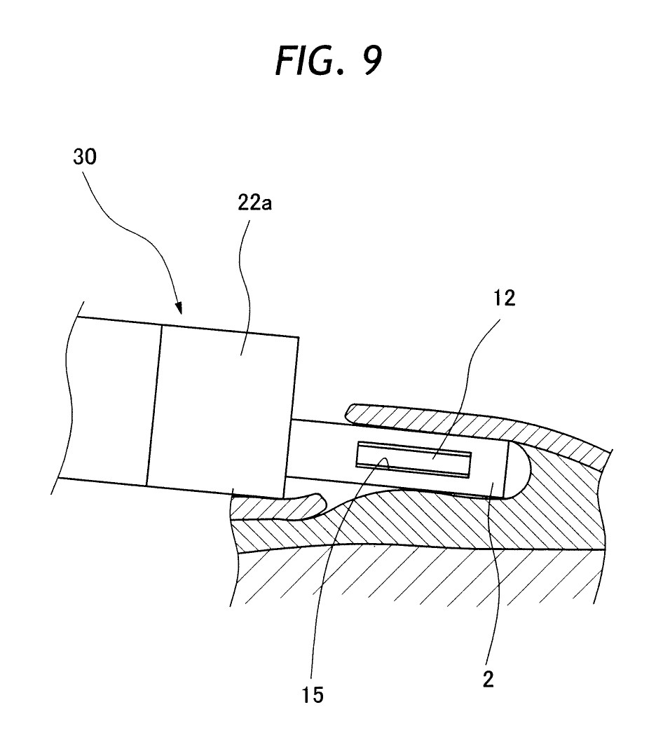

At the dissection, the electrode 12 is projected from the distal end of the flexible sheath 2, but in the mucous ablation, as shown in FIG. 8, the electrode 12 is positioned at the receded position retracted into the flexible sheath 2. That is, by operating the slider 4b of the operating means 4, the electrode 12 constituting the treatment instrument main body 10 is retracted into the axial passage hole 5. By means of this operation, as shown in FIG. 9, most parts of the electrode 12 is located inside the lateral opening 15, and only a small length at the distal end portion is arranged at a front position from the lateral opening 15. As a result, the most parts of the electrode 12 are faced in the lateral opening 15. Here, the electrode 12 is formed by metal made of a rigid body and even if the electrode is partially located inside the lateral opening 15, because that both end portions are regulated by wall faces of the axial passage hole 5, it is held in a stable state. In this state, the treatment instrument main body 10 and the high frequency power source are held in a disconnected state except that a treatment is actually performed.

In the mucous ablation, the lateral opening 15 is opposed to the portion to be ablated, and the treatment instrument main body 10 and the high frequency power source are connected to each other. The fibers constituting the submucosal layer is ablated when the submucosal layer, which is a flexible member, is pressed onto the lateral opening 15, the electrode 12 exposed from the lateral opening 15 is brought into contact with the submucosal layer, and the high frequency current flows. By swinging of the bent portion 22b in the insertion portion 22 of the endoscope 20 as appropriate, the mucous membrane can ablate rapidly and smoothly. In the mucous ablation, the electrode 12 does not project from the distal end portion of the flexible sheath 2 but partially exposed inside the lateral opening 15. Since the submucosal layer is the most flexible portion to enter easily into the lateral opening 15, cauterization can be performed efficiently. However, as long as the electrode 12 keeps in the receded state, the other tissues, particularly of a muscle layer having some degree of tension and shape keeping property, do not go into the lateral opening 15. Therefore, during the operation, the electrode 12 does not contact directly with the muscle layer, thus ensuring safe operation and removing damage to the muscle layer or the like at the time of the mucous ablation.

Bleeding may occur at a treating spot during performing the mucous ablation or dissection. View of the treated area from the observation portion of the endoscope 20 becomes unclear depending on the degree of bleeding. Thus, by connecting washing liquid supply means to the liquid supply port 9 provided in the connecting member 3, physiological saline solution, for example, is supplied with a high pressure. As a result, since the physiological saline solution is injected through the injection port 6a at the distal end of the fluid passage 6, the bleeding portion can be washed out rapidly and efficiently. And by pressing the electrode 12 onto the bleeding portion and by flowing the high frequency current, the bleeding portion is coagulated and stanched.

Also, in an endoscopic inspection, if a mucous surface is fouled, accurate endoscopic inspection cannot be performed, and whether or not a lesion portion is present in the mucous membrane cannot be accurately detected. Moreover, in order to confirm the marking made by cauterization at dissection, the mucous surface should be in a clean state. For that purpose, the physiological saline solution is supplied.

The fluid passage does not necessarily have to be provided in the high frequency treatment instrument 1 as in the case where the inside of the body cavity can be washed by using washing-liquid supply means in another system or the like. Therefore, provision of the fluid passage 6 in the flexible sheath 2 is not indispensable. If the fluid passage 6 is not provided, the axial passage hole 5 in the flexible sheath 2 can be arranged to pass through the axis center.

In the mucous dissection, the length when the electrode 12 is projected the most from the distal end of the flexible sheath 2 should be larger than the thickness of the mucous membrane, but it should be smaller than the total of the mucous membrane and the submucosal layer. Also, in the mucous ablation performed with the electrode 12 located at the portion of the lateral opening 15, the electrode 12 needs to be positioned with the distal end located slightly front of the lateral opening 15.

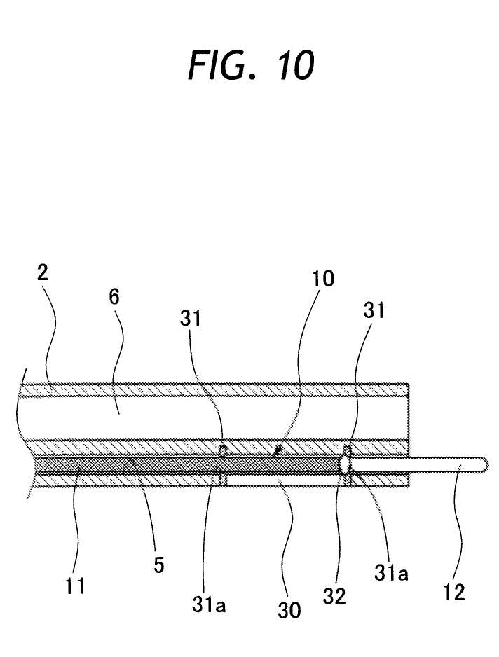

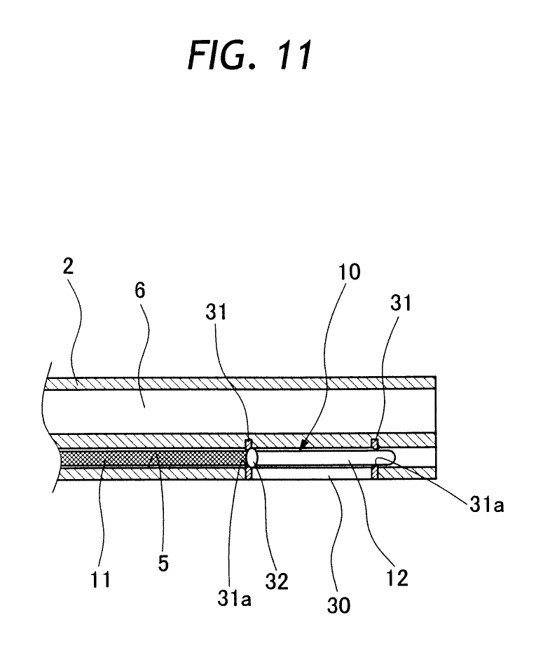

A restricting means for the stroke range to move the slider 4b or specifically, a stopper may be provided on the operating means 4. However, for the sake of more accurately the restriction of the stroke for the electrode 12, the restriction of the moving stroke can be performed by constructing that the positioning is performed at both positions of the operating position and the receded position at a portion on the distal end side of the flexible sheath 2. Such a construction can be adopted as shown in FIGS. 10 and 11 for this purpose.

That is, the lateral opening 30 for exposing the electrode 12 is formed in the flexible sheath 2 in which the biopsy channel 5 is provided, and positioning chips 31 and 31 are attached to both end portions in the front and rear of the lateral opening 30. The positioning chip 31 has a throttle portion 31a formed within the axial passage hole 5 through which the treatment instrument main body 10 is to be inserted, and the flexible cord 11 and the electrode 12 have a dimension capable of passing through the throttle passage 31a. A stopper projection 32 having larger diameter than the throttle passage 31a is formed on the border portion between the flexible cord 11 and the electrode 12. The stopper 32 is disposed between the both positioning chips 31 and 31.

By so constructed, when the treatment instrument main body 10 is moved in order to be protruded or retracted by the operation of the operating means 4, the stopper 32 is brought into contact with either of the throttle portions 31a and restricted further movement. As a result, by moving the treatment instrument main body 10 to the operating position shown in FIG. 10 and the receded position shown in FIG. 11, the treatment instrument main body maintains at these positions stably. Therefore, in the mucous ablation, if the electrode 12 keeps at the protruded state by a predetermined length, thereby the electrode 12 being held at its position stably. Also, when being located at the receded position, the electrode 12 is fully retracted into the flexible sheath 2 and made stable in a state facing in the lateral opening 30. As a result, smooth and efficient treatments can perform by using the electrode 12 such as mucous dissection and ablation smoothly and efficiently, and safety of the operation is further improved.

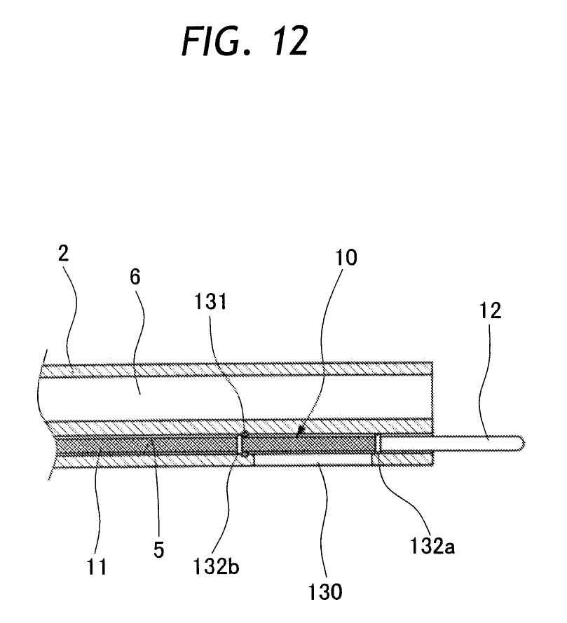

In this connection, the positioning chips 31 and the stopper 32 can be placed more retracted positions from the lateral opening 30 so as to not become expose the stopper 32 at the lateral openings 30. Further, FIG. 12 is another embodiment of the restricting means for the stroke range of the electrode 12. This embodiment has one positioning chips 131 within the lateral opening 30 and two stoppers 132a, 132b provided on the treatment instrument main body 10. The stopper 132a has a position of the fore side of the treatment instrument main body 10, while being placed the other stopper 132b at the rear side. The fore side stopper 132a is formed not to protrude from the distal end of the flexible sheath 2 at a position of the operating position by abutting against the positioning chip 131. On the other hand, the rear side stopper 132b is mounted on the electrode 12 at a position apart from the full stroke range from the fore side stopper 132a, thereby the electrode 12 being disposed within the lateral opening 30 when it is retracted at the receded position. Further, two positioning chips can be provided in the axial passage hole 5 so as to be engaged independently the fore side stopper and the rear side stopper.

What is claimed is:

1. A high frequency treatment instrument comprising
a flexible sheath having an axial passage hole;
an operating means connected to a proximal end portion of said flexible sheath;
a treatment instrument main body consisting of a flexible cord and an electrode provided at a distal end of said flexible cord to be inserted into said axial passage hole of said flexible sheath;
said electrode being displaced between a retracted position in said flexible sheath and a protruded position out of a distal end by push or pull operation of said flexible cord by said operating means;
said flexible cord consisting of a conductive body covered by an electrical insulating body and said electrode being electrically connected to said conductive body; and
a lateral opening formed at a lateral surface of said flexible sheath;
whereby a part of said electrode being exposed in said lateral opening at said retracted position, while said electrical insulating member of said flexible cord being faced with said lateral opening at said protruded position.

2. The high frequency treatment instrument according to claim 1, wherein a fore side of said lateral opening is placed a rear side of a distal end of said electrode at said retracted position.

3. The high frequency treatment instrument according to claim 1, wherein said lateral opening has a width dimension smaller than a diameter of said electrode and the entire length thereof is shorter than the length of said electrode.

4. The high frequency treatment instrument according to claim 1, wherein said axial passage hole of said flexible sheath has a first throttle portion at a rear side position of said lateral opening and a second throttle portion at a fore side position of said lateral opening, and a stopper member is mounted on said treatment instrument main body to engage with said first throttle portion at said retracted position of said electrode, while to engage with said second throttle portion at said protruded position of said electrode.

5. The high frequency treatment instrument according to claim 1, wherein said stopper member is mounted on said treatment instrument main body at a joint position of said electrode and said flexible cord.

6. The high frequency treatment instrument according to claim 1, wherein a liquid supply passage hole formed in said flexible sheath lateral to said axial passage hole.

7. The high frequency treatment instrument according to claim 1, wherein said flexible sheath having a diameter smaller than a biopsy channel of an endoscope.
